(19) United States
(12) Patent Application Publication
(10) Pub. No.: US 2003/0153065 A1
(43) Pub. Date: Aug. 14, 2003

(54) Composition and method for maintaining non-enveloped viral vectors

(75) Inventors: Imre Kovesdi, Rockville, MD (US); Stephen C. Ransom, Gaithersburg, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: GenVec, Inc., Gaithersburg, MD

(21) Appl. No.: 10/046,517

(22) Filed: Jan. 14, 2002

(51) Int. Cl. C12N 7/01

(52) U.S. Cl. 435/235.1

(57) ABSTRACT

The invention provides a composition and a method for preserving a non-enveloped viral vector. The composition comprises (a) trehalose, (b) a divalent metal salt, a cationic polymer, or a combination thereof, (c) a multiplicity of non-enveloped viral vector particles, and (d) a liquid carrier. Non-enveloped virus particles are stable in the composition in a liquid form, at elevated temperatures, for a sustained period of time.

### TECHNICAL FIELD OF THE INVENTION

The invention relates to compositions and methods for the preservation of non-enveloped viruses.

### BACKGROUND OF THE INVENTION

Viruses (modified and unmodified) have several applications in modem biology wherein preservation (maintenance or storage) of the virus (for example, in a virus stock or other composition comprising a virus) is desirable. Viral vectors, i.e., viruses that can comprise heterologous gene sequences, for example, have proven convenient systems for investigative and therapeutic gene transfer applications. The use of viral vectors in such investigative and therapeutic applications necessitates that the viral vectors be transported and stored for a period of time. During this period of storage, the viral vectors desirably are maintained without significant loss of infectivity and viability (activity). Viruses are useful in other contexts, such as the production of an immune response to the virus in the case of an inactivated viral vaccine. In such contexts, preservation of the virus typically does not require retention of infectivity and/or viability of the virus, but rather the storage method can (and often seeks to) maintain (and even sometimes cause) the virus to be inactivated and/or attenuated, but stored in a manner wherein the immunogenicity of the virus particle is retained.

The preservation of viruses, including active viruses, at very low temperatures (e.g., −80° C.) without significant loss of activity is known; however, the need for low temperature freezers, which are not widely available, limits the practicality of this approach. Lyophilization, or freeze-drying, is another known technique for storing viruses (see, e.g., Cryole et al., Pharm. Dev. Technol., 3(3), 973-383 (1998)). Lyophilization has disadvantages as it is expensive, and, upon reconstitution, the virus composition is often left for extended periods of time at room temperature (i.e., 20-25° C.). In storage formulations presently known in the art, active viruses rapidly lose viability when stored at room temperature.

Recently, several attempts have been made to provide liquid formulations useful for storing and administering viruses. International Patent Application WO 99/41416 discloses a liquid composition for preserving viral vectors comprising a “polyhydroxy compound,” which can be a disaccharide, such as sucrose. U.S. Pat. No. 6,165,779 discloses compositions comprising a recombinant viral vector, a buffer, a detergent, and a stabilizing agent such as glucose, sucrose, or dextran. However, such compositions are not entirely desirable for maintaining a suitable level of viral activity and infectivity.

U.S. Pat. No. 6,255,289 and related International Patent Application WO 00/34444 disclose liquid compositions comprising adenovirus particles and a stabilizer selected from the group of polysorbate 80, L-arginine, polyvinylpyrrolidone, trehalose, and combinations thereof. The compositions of the '289 patent and '444 application are capable of maintaining a high percentage of active viral vectors in a liquid composition. However, the inventors of the subject matter described in the '289 patent and '444 application now have discovered an even more effective composition for preserving non-enveloped viruses, which is the subject of the invention described herein. These and other advantages of the invention, as well as additional inventive features, will be apparent from the description of the invention provided herein.

### BRIEF SUMMARY OF THE INVENTION

The invention provides a composition for maintaining a non-enveloped viral vector and a method of using the composition to preserve and/or administer a non-enveloped viral vector to a host cell. The composition comprises (a) trehalose, (b) a divalent metal salt, a cationic polymer, or a combination thereof, (c) a multiplicity of non-enveloped viral vector particles, and (d) a liquid carrier. Preferably, the components of the composition are such that a substantial portion of the viral vector particle activity of the composition is retained over a sustained period of time.

### DETAILED DESCRIPTION OF THE INVENTION

The invention provides a composition and method for maintaining a non-enveloped viral vector in a liquid state while retaining a significant proportion of stable, infective, and active viral vector, even over an extended period of time (e.g., about 6 months, about 1 year, or longer). The composition comprises (a) trehalose, (b) a divalent metal salt, a cationic polymer, or a combination thereof, (c) a multiplicity of non-enveloped viral vector particles, and (d) a liquid carrier.

Trehalose (α-D-glucopyranosyl α-D-glucopyranoside dihydrate) is known in the art and described in, for example, U.S. Pat. Nos. 6,225,289 and 4,891,319. The composition comprises typically about 1% or more (wt./vol.) trehalose and more typically and preferably about 2% or more (wt./vol.) trehalose (e.g., about 3% or more (wt./vol.) trehalose or about 4% or more (wt./vol.) trehalose). The composition also comprises typically about 25% or less (wt./vol.) trehalose and more typically and preferably about 20% or less (wt./vol.) trehalose (e.g., about 15% or less (wt./vol.) trehalose, about 10% or less (wt./vol.) trehalose, or about 6% or less (wt./vol.) trehalose).

The divalent metal salt can be any suitable divalent metal salt. Suitable divalent metal salts include, for example, calcium chloride, magnesium chloride, and magnesium sulfate. Two or more divalent metal salts can be present in the composition. The preferred divalent salt is a magnesium salt, such as magnesium chloride or magnesium sulfate. Magnesium chloride (MgCl₂) is especially preferred; however, it has been reported that MgCl₂ may have a destabilizing effect on some viruses (see, e.g., Wallis et al., J. Bacteriol., 91(5), 1932-1935 (1966), and Habili et al., Virol,. 60, 29-36 (1974)). Accordingly, if the composition comprises viruses undesirably destabilized by MgCl₂, then the divalent metal salt preferably is magnesium sulfate or a non-magnesium divalent metal salt.

A cationic polymer in lieu of, or in conjunction with, a divalent metal salt can be present in the composition. The cationic polymer can be any suitable cationic polymer. Two or more cationic polymers can be present in the composition. Examples of suitable cationic polymers include, but are not limited to, polylysine, polyethyleneimine, polytrimethylaminoethyl methacrylate, poly(4-vinylpyridinium), diethylaminoethyl (DEAE)-dextran, poly(acrylic acid), poly(amidoamine), poly(N-(2-hydroxypropyl)methylacrylamide), poly(dimethylaminoethyl methylacrylate), polyethylene glycol, poly(N-ethyl-4-vinyl pyridinium bromide), poly(trimethylammonioethyl methacrylate chloride), poly(vinylalcohol), poly(N-ethyl-4-vinylpyridinium bromide), and polyvinylsulfonate.

The composition comprises desirably about 0.05 mM or more divalent salt(s) and/or cationic polymer(s) (e.g., about 0.1 mM or more, about 0.2 mM or more, about 0.5 mM or more, about 0.7 mM or more, or about 0.9 mM or more divalent salt(s) and/or cationic polymer(s)). The composition comprises typically about 2 mM or less divalent metal salt(s) and/or cationic polymer(s) (e.g., about 1.5 mM or less, about 1.3 mM or less, about 1.2 mM or less, about 1.1 mM or less, or about 1 mM or less divalent salt(s) and/or cationic polymer(s)). The divalent metal salt(s) and/or cationic polymer(s) can be added to the composition in any form to obtain the desired concentration in the composition. For example, a desired MgCl₂ concentration can be obtained by the addition to the composition of MgCl₂ hexahydrate, which is convenient for storage and handling.

The non-enveloped viral vector particles can be any suitable non-enveloped viral vector particles (i.e., particles of one or more different non-enveloped viral vectors). The non-enveloped viral vector particles can be infective and/or non-infective. Similarly, the non-enveloped viral vector particles can be active and/or inactive. The non-enveloped viral vector particles preferably are infective and preferably are active.

The non-enveloped viral vector particles can be particles of wild-type non-enveloped viruses and/or modified non-enveloped viruses (e.g., non-enveloped viral gene transfer vectors). Suitable non-enveloped viruses include, but are not limited to, reoviruses, adenoviruses, adeno-associated viruses, papovaviruses, parvoviruses, picornaviruses, and enteroviruses of any suitable origin (preferably of animal origin (e.g., avian or mammalian) and desirably of human origin). Other suitable non-enveloped viruses are known in the art and are well characterized. Examples of such non-enveloped viruses are described in, for example, Fields et al., VIROLOGY Lippincott-Raven (3rd ed. (1996) and 4th ed. (2000)); ENCYCLOPEDIA OF VIROLOGY, R. G. Webster et al., eds., Academic Press (2nd ed., 1999); FUNDAMENTAL VIROLOGY, Fields et al., eds., Lippincott-Raven (3rd ed., 1995); Levine, “Viruses,” Scientific American Library No. 37 (1992); MEDICAL VIROLOGY, D. O. White et al., eds., Academic Press (2nd ed. 1994); and INTRODUCTION TO MODERN VIROLOGY, Dimock, N. J. et al., eds., Blackwell Scientific Publications, Ltd. (1994).

The non-enveloped viral vector can comprise single-stranded or double-stranded DNA or RNA. Preferably, though not necessarily, the viral vector is derived from, or based on, a virus that normally infects animals, such as mammals (most preferably humans). Adenoviral vectors and adeno-associated viral (AAV) vectors based on human adenoviruses and AAV, respectively, are preferred non-enveloped viral vectors. Most preferably, the non-enveloped viral vector is an adenoviral vector.

Adenoviral vectors can be constructed and/or purified using the methods set forth, for example, in Graham et al., Mol. Biotechol., 33(3), 207-220 (1995), U.S. Pat. Nos. 5,922,576, 5,965,358 and 6,168,941, and International Patent Applications WO 98/22588, WO 98/56937, WO 99/15686, WO 99/54441, and WO 00/32754. Adeno-associated viral vectors (AAV vectors) can be constructed and/or purified using the methods set forth, for example, in U.S. Pat. No. 4,797,368 and Laughlin et al., Gene, 23, 65-73 (1983), Smith-Arica et al., Curr. Cardiol. Rep., 3(1), 43-9 (2001), Rabinowitz and Samulski, Virology, 278(2), 301-8 (2000), and Athanasopoulos et al., Int. J. Mol. Med., 6(4), 363-75 (2000).

The non-enveloped viral vector preferably is deficient in at least one gene function required for viral replication, thereby resulting in a “replication-deficient” viral vector. AAV vectors advantageously are naturally replication-deficient, requiring a complementation cell or helper virus providing adenovirus gene function for replication. Adenoviral vectors need to be made replication-deficient by deletion of one or more gene functions in one or more regions, such as the E1 region (e.g., the E1a region and/or the E1b region), E2 region, and/or E4 region of the adenoviral genome. Replication-deficient adenoviral vectors are described in U.S. Pat. Nos. 5,851,806, 5,994,106, and 6,136,594 and International Patent Applications WO 95/34671 and WO 97/21826. Replication-deficient adenoviral vectors can be produced by use of a complementation cell line, or helper virus, which is capable of providing the deleted necessary adenoviral gene functions in trans. Suitable adenovirus packaging cells are known and include 293 cells (described in, e.g., Graham et al., J. Gen. Virol., 36, 59-72 (1977)), HER cells, such as 911 cells (as described in, e.g., Fallaux et al., Hum. Gene Ther., 7, 215-222 (1996)) or PER.C6 cells (commercially available through Crucell (Leiden, Netherlands)), and 293-ORF6 cells (as described in, e.g., International Patent Application WO 95/34671 and Brough et al., J. Virol., 71, 9206-13 (1997)).

The non-enveloped viral vector can be subject to any number of additional or alternative modifications. For example, an adenoviral vector may be a replication-deficient adenoviral vector which includes or produces (by expression) a modified adenoviral protein, non-adenoviral protein, or both, which increases the efficiency with which the vector infects cells as compared to wild-type adenovirus, allows the vector to infect cells which are not normally infected by wild-type adenovirus, results in a reduced host immune response in a mammalian host as compared to wild-type adenovirus, or any combination thereof. Such modifications can be effected by modifying the viral coat proteins (e.g., the adenoviral fiber, penton, pIX, pIIIa, or hexon proteins) and/or by inserting various native or non-native ligands into portions of the viral coat proteins. Viral vector modifications are described in Miller et al., FASEB J., 9, 190-99 (1995), Douglas et al., Nat. Biotechnol., 14(11), 1574-78 (1996), Wickam, Gene Ther., 7(2), 110-14 (2000), U.S. Pat. Nos. 5,559,099, 5,712,136, 5,731,190, 5,770,442, 5,846,782, 5,962,311, 5,965,541, 5,985,655, 6,030,954, 6,057,155, 6,127,525, and 6,153,435 and International Patent Applications WO 96/07734, WO 96/26281, WO 97/20051, WO 98/40509, WO 98/07865, WO 98/07877, WO 98/40509, WO 98/54346, WO 00/15823, WO 00/34496, and WO 01/58940.

The non-enveloped viral vector can comprise a heterologous nucleotide sequence (also referred to herein as a transgene) encoding a gene product. The heterologous nucleotide sequence can exert an effect on a host cell at the RNA or protein level. The gene product can be, for example, an antisense molecule, a nucleozyme (e.g., a ribozyme or a ribonucleoprotein), or a protein. The gene product desirably is a protein, especially a protein that confers a prophylactic or therapeutic benefit to a cell or an animal containing the cell. Such a protein can be, for example, a vascular endothelial growth factor (e.g., VEGF₁₂₁ or VEGF₁₆₅), tumor necrosis factor (e.g., TNF-α), atonal-associated factor (e.g., Hath-1), pigment epithelium-derived factor (e.g., PEDF), or nitric oxide synthase (e.g., iNOS). The protein also can be a protein that affects splicing or 3′ processing (e.g., polyadenylation), or a protein that affects the level of expression of another coding sequence within the cell (i.e., where coding sequence expression is broadly considered to include all steps from initiation of transcription through production of a process protein), such as by mediating an altered rate of mRNA accumulation or transport or an alteration in post-transcriptional regulation.

The heterologous nucleotide sequence can be positioned in any suitable location in the genome of the non-enveloped viral vector. For example, if the non-enveloped viral vector is an adenoviral vector, the heterologous nucleotide sequence can substitute for one or more of the regions typically deleted in a replication-deficient adenoviral vector (e.g., the E1, E2, E3, and/or E4 region, most preferably replacing at least a portion of the E1 region). The non-enveloped viral vector can comprise other heterologous nucleotide sequences, which may or may not be expressed, such as regulatory sequences (e.g., promoters, enhancers, and polyadenylation sequences) operatively linked to the heterologous nucleotide sequence encoding the gene product, so as to allow for expression of the heterologous nucleotide sequence encoding the gene product in a host cell and production of the gene product. In that respect, the heterologous nucleotide sequence encoding the gene product typically will be part of a suitable expression cassette comprising non-expressed regulatory sequences, such as a promoter (e.g., a constitutive promoter such as the CMV promoter, or an inducible promoter such as a metallothionein promoter or EGR promoter), an enhancer, and a polyadenylation region (e.g., an SV40 polyA region).

The composition can include any suitable concentration of non-enveloped viral vector particles. Desirably, the composition comprises non-enveloped viral vector particles in a concentration of at least about 1×10⁵ particles/ml. Preferably, the composition comprises non-enveloped viral vector particles in a concentration of about 1×10⁵ particles/ml to about 1×10¹³ particles/ml. Most preferably, the composition comprises non-enveloped viral vector particles in a concentration of about 1×10⁶ particles/ml to about 1×10¹² particles/ml (e.g., about 1×10⁷ particles/ml to about 1×10⁹ particles/ml or about 1×10⁹ particles/ml to about 1×10¹² particles/ml). The number of particles can be measured in terms of particle forming units (PFU) or, more preferably, in terms of focus forming units (FFU). The number of particles can be measured by any suitable technique, such as electron microscopy, HPLC, and, preferably, UV spectrophotometry.

The liquid carrier can be any suitable carrier that is liquid at the ambient conditions of the use of the composition, typically 25° C. The liquid carrier desirably does not significantly impact the stability, infectivity, and/or gene expression activity of the non-enveloped viral vector particles. Preferably, the liquid carrier is a pharmaceutically (e.g., pharmacologically or physiologically) acceptable liquid carrier, particularly when the composition is a pharmaceutical composition. The liquid carrier most preferably is water. The liquid carrier can contain a buffer (e.g., a tris buffer) and a salt. Suitable liquid carriers (as well as buffers and salts therefore) are described in, e.g., Urquhart et al., Lancet, 16, 367 (1980), Lieberman et al., PHARMACEUTICAL DOSAGE FORMS—DISPERSE SYSTEMS (2nd ed., vol. 3, 1998), Ansel et al., PHARMACEUTICAL DOSAGE FORMS & DRUG DELIVERY SYSTEMS (7th ed. 2000), Martindale, THE EXTRA PHARMACOPEIA (31st edition), Remington's PHARMACEUTICAL SCIENCES (16th-20th editions), THE PHARMACOLOGICAL BASIS OF THERAPEUTICS, Goodman and Gilman, Eds. (9th ed.-1996), WILSON AND GISVOLDS TEXTBOOK OF ORGANIC MEDICINAL AND PHARMACEUTICAL CHEMISTRY, Delgado and Remers, Eds. (10th ed.-1998), Berge et al., J. Pharm. Sci., 66(1), 1-19 (1977), Wang and Hanson, J. Parenteral. Sci. Tech., 42, S4-S6 (1988), and U.S. Pat. Nos. 5,708,025, 5,994,106, 6,165,779, and 6,225,289. The preparation of pharmaceutically acceptable compositions is described in, e.g., Platt, Clin. Lab Med., 7, 289-99 (1987), Aulton, PHARMACEUTICS: THE SCIENCE OF DOSAGE FORM DESIGN, Churchill Livingstone (New York) (1988), EXTEMPORANEOUS ORAL LIQUID DOSAGE PREPARATIONS, CSHP (1998), and “Drug Dosage,” J. Kans. Med. Soc., 70(1), 30-32 (1969).

The composition optionally further comprises one or more nonionic surfactants. The nonionic surfactant can be any suitable nonionic surfactant. Desirably, the nonionic surfactant promotes the infectivity of the non-enveloped viral vector particles, reduces the amount or frequency of non-enveloped viral vector particle aggregation in the composition, or both, while not adversely impacting non-enveloped viral vector particle stability or activity of the non-enveloped viral vector particles during storage of the composition. Suitable nonionic surfactants include, for example, NP-40, Brij detergents, zwitterionic detergents such as CHAP detergents, octylphenoxypolyethoxy-ethanol (Triton X-100), C12E8, octyl-β-D-glucopyranoside, pluronic surfactants such as Pluronic F68, and polysorbate 20. A preferred nonionic surfactant is polysorbate 80 (also known as polyoxyethylene (20) sorbitan monooleate, Tween 80, and PEG-3/6 sorbitan oleate). Polysorbate 80 exhibits stabilizing effects on non-enveloped viral vectors both in the presence and absence of trehalose and in the presence of divalent metal salts, cationic polymers, or a combination thereof.

The non-ionic surfactant can be present in the composition in any suitable amount. Typically, the composition comprises a nonionic surfactant in a concentration of at least about 0.001% (wt./vol.). Desirably, the composition comprises a nonionic surfactant in a concentration of about 0.001-0.015% (wt./vol.). Preferably, the nonionic surfactant is in a concentration of about 0.0015% (wt./vol.) to about 0.01% (wt./vol.), and more preferably about 0.0018% (wt./vol.) to about 0.007% (wt./vol.). Even more preferably, the composition comprises a nonionic surfactant in a concentration of about 0.002% (wt./vol.) to about 0.005% (wt./vol.). Ideally, the composition comprises a nonionic surfactant in a concentration of about 0.0025% (wt./vol.).

The composition optionally further comprises arginine. Arginine can promote the stability, infectivity, and/or activity of the non-enveloped viral vector particles in the composition. The composition can comprise any suitable amount of arginine. The composition desirably comprises about 10 mM or more arginine, preferably 30 mM or more arginine, although usually about 65 mM or less arginine (e.g., about 10-65 mM arginine or about 30-65 mM arginine). More preferably, the concentration of arginine in the composition is about 25-55 mM. Most preferably, the concentration of arginine in the composition is about 30-50 mM (e.g., about 40 mM).

The composition can comprise other components. Such other components include, for example, buffers, salts, diluents, pH adjusters, and the like.

The composition can have any suitable osmolality (concentration of particles). The composition desirably has an osmolality within the range of about 150-800 mOsM. Compositions that do not have an osmolality within this range may be relatively less effective at stably preserving non-enveloped viral vector particles. The osmolality of the composition is preferably about 200 mOsM or more, more preferably about 300 mOsM or more. Moreover, the osmolality of the composition is preferably about 700 mOsM or less, more preferably about 600 mOsM or less, and yet more preferably about 500 mOsM or less. Most preferably, the composition has an osmolality of about 300-500 mOsM.

The composition can have any suitable ionic strength and desirably has an ionic strength that promotes the stability, infectivity, and/or activity of the viruses therein. The ionic strength of the composition is desirably about 10 mM or more, preferably about 50 mM or more (e.g., about 60 mM or more, or about 70 mM or more). Moreover, the ionic strength of the composition is desirably about 250 mM or less, preferably about 200 mM or less, and more preferably about 150 mM or less (e.g., about 130 mM or less, about 110 mM or less, or about 100 mM or less). Most preferably, the composition has an ionic strength of about 70-100 mM. For the definition and manner of determining the ionic strength of a composition, see Atkins, Physical Chemistry (5^{th} edition), p. 321 (W. H. Freeman and Co., New York, 1994).

The ionic strength of the composition can be adjusted with the use of one or more ionic salts and/or diluents. Any suitable ionic salts and/or diluents can be used to achieve the desired ionic strength.

Suitable ionic salts include monovalent salts, divalent salts, and polyvalent salts that comprise one or more cations selected from Group I elements, Group II elements, and Group III elements, polyatomic cations, and one or more counteranions. Polyatomic cations include, for example, ammonium, alkylammonium, and dialkylammonium. Counteranions include, for example, chloride, iodide, sulfate, phosphate, acetate, cabonate, oxolate, succinate, and fluoride. The salt desirably is water-soluble. Preferably, the salt is a monovalent or divalent salt. Monovalent salts are most preferred. Particularly useful are sodium salts and halides. More particular examples of useful salts include MgSO₄ and CaCl₂. Most desirably, the ionic salt is sodium chloride (NaCl). The concentration of the divalent metal salt of the composition (described elsewhere herein) also can be adjusted to obtain the desired ionic strength (although it is preferred that the amount of divalent metal salt(s) not adversely affect the desirable properties of the composition).

Suitable diluents are well known in the art, and include, for example, water and short-chain alkyl alcohols. Such alcohols can be present in the composition in any suitable amount, e.g., up to about 20% (wt./vol.). Preferably, the concentration of alcohol in the composition is about 15% (wt./vol.) or less. More preferably, the concentration of alcohol in the composition is about 12.5% (wt./vol.) or less, and, still more preferably, the concentration of alcohol in the composition is about 10% (wt./vol.) or less.

The composition can have any suitable pH. The composition preferably has a pH that maintains the viral vector particle stability and, more preferably, viral vector particle infectivity and/or activity of the composition, for a desired period of time (e.g., at least about 1 day, at least about 3 days, at least about 1 week, at least about 1 month, or longer). To maintain a desired pH, the composition typically comprises at least one buffer, such that the pH of the composition is adjusted to, and maintained at, about pH 6-9 when the temperature of the composition is about 1-50° C. Preferably, the composition has a pH of about 7-8.5, more preferably about 7.5-8, and most preferably about 7.8.

Any suitable buffer can be used to stabilize the composition's pH. Suitable buffers include phosphate buffered saline (PBS), sodium phosphate, sodium sulphate, Tris buffers, glycine buffer, and sterile water. Particularly desirable buffers include Tris-HCl and phosphate buffers. Formulation with a tris buffer to a pH of about 7.5-8.5 at room temperature (e.g., about 25° C.), for example, is desirable inasmuch as tris buffers are commonly associated with drops in pH at elevated temperatures (e.g., about 28° C. or about 37° C.), such that the composition is maintained within a desirable range of pH (e.g., at least about 7) across such a range of temperatures (e.g., when the composition is administered to a host). The choice of buffer will depend on the intended use of the composition. For example, PBS buffered compositions are useful for administration to peripheral tissues and organs (e.g., for the treatment of peripheral vascular disease), but are not desirable for administration to the heart.

The buffer can be present in the composition of any suitable concentration. Typically, the composition comprises a buffer in a concentration of about 5-100 mM. Preferably, the composition comprises a buffer in a concentration of about 5-75 mM (e.g., 50 mM). Even more preferably, the concentration of buffer in the composition is about 5-30 mM. Still more preferably, the concentration of buffer in the composition is about 5-20 mM (e.g., about 10 mM). The buffered composition's pH also can be adjusted by addition of any suitable acid or base (e.g., HCl).

The composition preferably maintains (e.g., preserves) the non-enveloped viral vector particles such that, over a desired period of time (e.g., about 48 hours, about 3 days, about 7 days (i.e., about 1 week), about 2 weeks, about 1 month, about 3 months, about 6 months, about 1 year, or about 2 years) at a desired temperature (e.g., about 0° C., about 5° C., about 10° C., about 15° C., about 20° C., about 25° C., about 30° C., about 35° C., about 40° C., about 45° C., or about 50° C.), the stability, infectivity, and/or activity of the non-enveloped viral vector particles in the composition is not significantly or substantially degraded or is retained to a significant extent (e.g., at least about 60%, at least about 70%, at least about 80%, at least about 90%, or at least about 95%). The retention (e.g., maintenance) of non-enveloped viral vector particle stability and activity is particularly preferred. In addition, the composition preferably minimizes the amount and/or frequency of non-enveloped viral vector particle aggregation. Non-enveloped viral vector particle stability, infectivity, activity, and aggregation can be determined by any suitable techniques. Many such techniques are known in the art.

The “stability” of the non-enveloped viral vector particles refers to the ability of the non-enveloped viral vector particles to maintain structural integrity over time. The stability of the non-enveloped viral vector particles is reflected in the structural integrity of the particles (e.g., how many “empty” or degraded capsids are in the composition). Suitable techniques for determining non-enveloped viral vector particle stability include, for example, fluorescence detection techniques, light scattering techniques, electron microscope studies, and differential centrifugation (e.g., CsCl-density gradient centrifugation). The stability of the non-enveloped viral vector particles can be important even when the non-enveloped viral vector is an inactive virus (e.g., a vaccine composition comprising inactive viruses), as the immunogenic properties of the virus often will depend on conformation-dependent viral antigens in the virus capsid.

The “infectivity” of the non-enveloped viral vector particles refers to the ability of the non-enveloped viral vector particles to infect cells. Suitable techniques for measuring non-enveloped viral vector infectivity include, for example, plaque formation assays and focus formation assays. In a standard plaque assay, a confluent monolayer of susceptible cells are provided and infected -with a composition comprising a quantified population of a virus, which displays a visible cytopathic effect (cell killing or cell damage). The cells are covered with a semisolid overlay (e.g., an agar covering layer), which prevents virus particle diffusion. As a result, discrete plaques are visualized by staining a cell with a suitable dye such as crystal violet or natural red. A focus-formation assay typically relies upon the use of antibody staining methods to detect virus antigens within infected cells in the monolayer. These infected cells then are visualized using a fluorescent label on the virus-specific antibody and counted. For example, permeabilized cells susceptible to infection with the virus are stained with fluorescein-conjugated monoclonal antibody against an early adenovirus nuclear protein (DNA-binding protein) for about 1 hour. After about 1 hour incubation, the staining conjugate is washed off, and the cells are visualized with an inverted fluorescence microscope. With appropriate illumination, the fluorescein dye emits a green wavelength of light, which is seen with the human eye under a microscope. Cells that have been infected with adenovirus have a fluorescent green nucleus because of the presence of DNA binding protein bound by the antibody conjugate. Only virus-infected cells stain with the conjugate, permitting an approximate determination of the number of active adenoviruses in the composition. Because the non-enveloped viral vector particles desirably do not lyse infected cells for a period of at least about 48 hours after infection, such fluorescent-focus assays are preferred to determine infectivity in the context of the invention.

Other suitable infectivity assays include infectious center assays, endpoint dilution assays, transformation assays, assays of the production of antiviral antibodies upon infection a cell population with a population of viruses (e.g., by using an ELISA or Western Blot assay), PCR assays (e.g., quantitative PCR assays such as the TaqMan assay system) directed to the number of viral nucleic acids (or viral particle transgene-associated nucleic acids) in a population of virus infected cells, and assays that measure the production of cytokines (e.g., interferons) generated in response to the introduction of the virus into a given host. In conducting infectivity assays, a suitable amount of time is afforded for viral infection of the cells prior to the determination of infectivity.

The “activity” of the non-enveloped viral vector particles refers to ability of the non-enveloped viral vector particles to express coding sequences to produce gene products within a host cell (e.g., a specific viral protein or RNA). Activity can be characterized by relative percent activity, total percent activity, or both. “Relative percent activity” refers to the amount of active non-enveloped viral vector particles in the composition at an end point in time as compared to the number of non-enveloped viral vector particles in the composition at a starting time. “Total percent activity” refers to the proportion of active non-enveloped viral vector particles in the composition as compared to the total number of active and inactive non-enveloped viral vector particles in the composition. When the non-enveloped viral vector is a viral gene transfer vector, activity desirably is a measure of the amount of heterologous nucleotide sequence-encoded gene product produced by cells infected by a sample of the non-enveloped viral vector particle composition.

Suitable techniques for measuring coding sequence expression and resulting gene product production include, for example, Northern Blot analyses (discussed in, e.g., McMaster et al., Proc. Natl. Acad. Sci. USA, 74, 4835-38 (1977), and Sambrook, infra), RT-PCR (as described in, e.g., U.S. Pat. No. 5,601,820 and Zaheer et al., Neurochem. Res., 20, 1457-63 (1995)), and in situ hybridization techniques (as described in, e.g., U.S. Pat. Nos. 5,750,340 and 5,506,098). To determine changes in viral gene expression activity, the femtograms of product produced per microliter of liquid composition per unit time can be determined under similar conditions at different test times. Reporter genes, such as β-galactosidase and green fluorescent protein genes, can be used to measure viral gene expression activity, desirably under conditions where reporter gene expression reflects levels of expression of a heterologous nucleotide sequence of interest (e.g., as part of a fusion protein or as part of an operably-linked expression cassette). Activity will necessarily correspond somewhat to the infectivity of the non-enveloped viral vector particle composition; however, infectivity can be a poor measure of activity. As viral gene transfer vector particles are preferred as the non-enveloped viral vector particles, retention of activity is a preferred characteristic of the composition. For activity testing, like infectivity testing, some time can be required to test viral particle-directed gene expression levels. The amount of time required will depend on the virus. In general, activity assays can be conducted a number of hours to a few days after infection has occurred.

The precise measurement technique for viral activity and/or infectivity will depend, to some extent, upon the particular composition, especially the particular non-enveloped viral vector particles therein (e.g., the nature of the viral gene transfer vector and product(s) produced thereby). Techniques and principles related to such assays are discussed further, for example, in Fields et al., supra, and Sambrook et al., MOLECULAR CLONING: A LABORATORY MANUAL (Cold Spring Harbor Press 1989) and the third edition thereof (2001)), and Ausubel et al., CURRENT PROTOCOLS IN MOLECULAR BIOLOGY (Wiley Interscience Publishers 1989 and 1995). Additional molecular biological techniques useful in the practice of the invention are described in, e.g., Watson et al., RECOMBINANT DNA, (2d ed.), Mulligan, Science 260, 926-932 (1987), Friedman, Therapy For Genetic Diseases (Oxford University Press, 1991), Ibanez et al., EMBO J., 10, 2105-10 (1991), Ibanez et al., Cell, 69, 329-41 (1992), and U.S. Pat. Nos. 4,440,859, 4,530,901, 4,582,800, 4,677,063, 4,678,751, 4,704,362, 4,710,463, 4,757,006, 4,766,075, and 4,810,648.

Suitable techniques for determining or evaluating the aggregation of non-enveloped viral vector particles include, for example, electron microscope examination, differential centrifugation, and light scattering techniques. These techniques typically are performed on an aliquot of the composition.

The composition of the invention can be prepared in any suitable manner. For example, a replication-competent adenovirus-free (RCA-free) stock of replication-deficient adenoviral vectors as described in U.S. Pat. No. 5,994,106 can be combined with the other components described herein to form the composition of the invention. Alternatively, for example, cells infected with the non-enveloped viral vector particles can be lysed with a nonionic surfactant in a concentration of about 0.5-2% (wt./vol.) to obtain a lysate, the lysate can be purified (e.g., by ion exchange chromatography (preferably anion exchange chromatography as described in International Patent Application WO 99/54441), filtration (such as microfiltration and/or tangential flow ultrafiltration), size exclusion chromatography, or a combination thereof), and the other components described herein can be added to form the composition of the invention. Of course, some of the other components of the composition of the invention, such as the divalent metal salt and/or cationic polymer, may be present in the RCA-free stock or lysing or purification formulations, such that further addition of certain of these other components to form the composition of the invention may not be necessary or only may be necessary to a more limited extent.

The invention also provides a method of preserving a non-enveloped viral vector comprising maintaining a multiplicity of non-enveloped viral vector particles in a composition of the invention for a period of about 48 hours (i.e., about 2 days), wherein at least about 50% of the non-enveloped viral vector particles in the composition are active at the end of the period as compared to the beginning of the period. Preferably, at least about 60% of the non-enveloped viral vectors in the composition remain active at the end of the period as compared to the beginning of the period, and more preferably at least about 70% of the non-enveloped viral vectors remain active at the end of the period as compared to the beginning of the period. Still more preferably, at least about 80% of the non-enveloped viral vectors in the composition remain active at the end of the period as compared to the beginning of the period. Ideally, at least about 90% (e.g., at least about 95%) of the non-enveloped viral vectors in the composition remain active at the end of the period as compared to the beginning of the period. The composition is maintained during the period at any suitable temperature, preferably a temperature at which the composition remains in liquid form (e.g., about 0° C., about 5° C., about 10° C., about 15° C., about 20° C., about 25° C., about 30° C., about 35° C., about 40° C., about 45° C., or about 50° C.), especially at about 25° C. Desirably, the composition is retained at a temperature below ambient temperature (especially at a refrigeration temperature, such as about 0-10° C. and preferably about 5° C.). The non-enveloped viral vectors in the composition preferably retain activity at the aforementioned levels after being maintained at the aforementioned temperatures for periods of time longer than about 48 hours, such as about 3 days, about 7 days (i.e., about 1 week), about 2 weeks, about 1 month, about 3 months, about 6 months, about 1 year, and about 2 years. The invention most preferably allows for no significant or substantial (if any) decrease in the activity of the non-enveloped viral vector particles in the composition at any of the aforementioned storage temperatures and for any of the aforementioned time periods, although some loss of activity is acceptable, especially with relatively higher storage temperatures and/or relatively longer storage times.

The composition can be maintained in any form that is, or can be reconstituted to be, a liquid at about 25° C.). Thus, for example, the composition can be prepared as a liquid composition and then stored as a solid at a freezing temperature (e.g., about −20° C., about −80° C., or less) for the aforementioned time periods. Moreover, the composition can form a gel or semisolid material or be used in the formation of a gel or semisolid material. Similarly, the composition can be freeze-dried and then reconstituted in liquid form for later use. Although such changes in form can be acceptable, the composition desirably remains in liquid form throughout its existence, i.e., without being changed into a solid form and then back again to a liquid form.

The invention further provides a method of administering a non-enveloped viral vector particle to a host cell comprising contacting a host cell with the liquid composition of the invention to infect the host cell with at least one non-enveloped viral vector particle. The composition can be administered to the host cell in vitro, in vivo, or ex vivo. In that respect, the host cell can be in a mammal (such as a human), or, for example, part of an organ (such as the heart), or otherwise present in the mammal (such as in a tumor in the mammal). The composition can contact the host cell by any suitable manner. The composition can be administered by a variety of routes. Local or systemic delivery can be accomplished by application or instillation into body cavities, by inhalation or insufflation of an aerosol, or by parenteral introduction, comprising intermuscular, intramuscular, intravenous, intraperitoneal, intraocular, transtympanical, transdermal, intemasal, or subcutaneous administration, or by other means. The composition can be delivered to a specific tissue, organ, gland, or other part of a human patient's body (e.g., a tumor, a limb such as the leg, the lungs, the brain, the eye, or the ear). The composition also can be administered to a tissue ex vivo or be used to infect cells in a tissue culture for in vitro studies or applications. The composition desirably is administered to cells so that the population of non-enveloped viral vector particles relative to the number of cells results in a multiplicity of infection (MOI) of about 1-100, more preferably an MOI of about 5-30.

### EXAMPLES

The invention is further described in the following examples. These examples serve only to illustrate the invention and are not intended to limit the scope of the invention in any way.

### Example 1

This example demonstrates the effectiveness of a composition comprising trehalose and a divalent metal salt, in accordance with the invention, with or without the addition of polysorbate 80, to maintain the gene expression activity of a non-enveloped viral vector for a period of up to at least a year.

Two compositions were prepared. Each composition contained sterile water, 10 mM Tris-HCl (pH 7.8 at room temperature (i.e., 20-25° C.)), 75 mM NaCl, 0.08 mM MgCl₂, and 5% (wt./vol.) trehalose. One of the two compositions also contained 25 ppm polysorbate 80, while the other of the two compositions did not contain any polysorbate 80.

An equal amount of E1/E3-deficient adenoviral vector particles comprising a secretory alkaline phosphatase (SEAP) transgene under control of the cytomegalovirus (CMV) promoter inserted in the E1 region (Ad.SEAP) was added to each composition. Each composition was stored in a plastic container at −20° C., 4° C., and 25° C. for varying amounts of time, and then the activity of the viral vector was determined. Activity was determined by measuring the amount of SEAP produced upon infection of A549 cells with a sample of each of the compositions. Experiments were repeated until a range of activity measurements was obtained. The results of these experiments are set forth in Table 1.

TABLE 1
Percent Transgene Expression Activity with Respect to Composition Storage Time

| Storage Temperature | −20° C. | | 4° C. | | 25° C. | |
|---|---|---|---|---|---|---|
| Polysorbate 80 added? | no | yes | no | yes | no | yes |
| Day 1 | 97 ± 12 | 100 ± 8 | 97 ± 3 | 104 ± 8 | 92 ± 3 | 94 ± 8 |
| Day 7 | 106 ± 10 | 107 ± 18 | 97 ± 10 | 99 ± 9 | 76 ± 9 | 92 ± 17 |
| Day 21 | 124 ± 10 | 120 ± 9 | 98 ± 5 | 93 ± 18 | 71 ± 4 | 73 ± 7 |
| Day 42 | 120 ± 12 | 106 ± 18 | 119 ± 7 | 108 ± 10 | 46 ± 4 | 54 ± 2 |
| Day 182 | 99 ± 6 | 114 ± 4 | 61 ± 9 | 83 ± 13 | nd | nd |
| Day 365 | 95 ± 4 | 97 ± 20 | 56 ± 8 | 67 ± 9 | nd | nd |

nd = not determined

As is apparent from the experimental results set forth in Table 1, the composition of the invention can maintain a population of non-enveloped viral vector particles with little decrease in transgene expression activity at freezing temperatures, refrigeration temperatures, and ambient temperatures for sustained periods of time. At a storage temperature of −20° C., less than a 20% loss of transgene expression levels was observed for the composition, with or without the presence of polysorbate 80, after 1 year of storage. At a storage temperature of 4° C., less than a 20% loss of transgene expression activity was observed for the composition, with or without the presence of polysorbate 80, after 6 weeks of storage. At a storage temperature of 25° C., less than a 30% loss of transgene expression activity was observed for the composition, with or without the presence of polysorbate 80, after 3 weeks of storage.

The experimental results set forth in Table 1 also demonstrate that the presence of polysorbate 80 in the composition increased the retention of transgene expression activity. The effect of the polysorbate 80 to increase transgene expression activity retention generally was more pronounced at higher storage temperatures. For example, the presence of polysorbate 80 in the composition was associated with almost a 20% increase in transgene expression activity after storage for 6 weeks at 25° C.

The results of these experiments demonstrate that the composition of the invention maintains a high level of non-enveloped viral vector transgene expression activity over a significant period of time at temperatures of −20° C. to 25° C.

### Example 2

This example illustrates the impact of ionic strength on the level of non-enveloped viral vector particle activity in a composition maintained in liquid form over a sustained period of time.

A liquid composition comprising 10 mM Tris (pH 7.8 at 37° C.), 0.08 mM MgCl₂, 3% (wt./vol.) sucrose, sterile water, and a population of Ad.SEAP particles, as described in Example 1, was prepared. An aliquot of the composition was taken and used to infect cells. The cells were incubated, and the SEAP expression level was determined as a control. Six similar additional aliquots of the composition, containing an equal amount of Ad.SEAP particles, were obtained. NaCl was added to the six aliquots to obtain viral vector particle compositions with the following NaCl concentrations: 20 mM, 50 mM, 75 mM, 100 mM, 125 mM, and 150 mM. The viral vector particle compositions were maintained at 37° C. for a period of seven days. At the end of the seven-day period, the compositions were used to infect A549 cells. The cells were incubated for a set period of time, SEAP expression levels were determined using standard techniques, and relative SEAP expression levels were calculated. The experiments were repeated to obtain a range of SEAP expression level values. The results of these experiments are set forth in Table 2.

TABLE 2
Relative Transgene Expression Activity After 7 Days at Various NaCl
Concentrations

| | 20 mM | 50 mM | 75 mM | 100 mM | 125 mM | 150 mM |
|---|---|---|---|---|---|---|
| | NaCl | NaCl | NaCl | NaCl | NaCl | NaCl |
| Relative % | 37 +/− 8 | 41 +/− 2 | 56 +/− 2 | 45 +/− 7 | 37 +/− 2 | 35 +/− 2 |
| Ad.SEAP | | | | | | |
| Activity | | | | | | |

As reflected in the experimental results set forth in Table 2, compositions comprising between about 50-100 mM ionic strength (NaCl) exhibited significantly higher levels of transgene expression activity after being maintained for a sustained period of time at 37° C. For example, the relative transgene expression activity observed for the composition comprising 75 mM NaCl was more than 150% greater than the relative transgene expression activity observed for the composition comprising 150 mM NaCl. Similarly high relative transgene expression activities were observed for compositions comprising 50 and 100 mM NaCl.

The results of these experiments demonstrate the effect of ionic strength in maximizing the retention of activity of the non-enveloped viral vector particles in a composition.

### Example 3

This example illustrates the importance of the divalent metal salt concentration on the retention of non-enveloped viral vector particle activity in a composition.

A liquid composition comprising 10 mM Tris (pH 7.8 at 37° C.), 20 mM NaCl, 3% (wt./vol.) sucrose, sterile water, and a population of Ad.SEAP particles, as described in Example 1, was prepared. An aliquot of the composition was taken and used to infect cells. The cells were incubated, and the SEAP expression level was determined as a control.

Six similar additional aliquots of the composition, containing an equal amount of Ad.SEAP particles, were obtained. MgCl₂ was added to the six aliquots to obtain viral vector particle compositions with the following MgCl₂ concentrations: 0.08 mM, 0.5 mM, 1 mM, 2 mM, 5 mM, and 10 mM. The viral vector particle compositions were maintained at 37° C. for a period of seven days. At the end of the seven-day period, the compositions were used to infect A549 cells. The cells were incubated for a set period of time, SEAP expression levels were determined using standard techniques, and relative SEAP expression levels were calculated. The experiments were repeated to obtain a range of SEAP expression level values. The results of these experiments are set forth in Table 3.

TABLE 3
Relative Transgene Expression Activity After 7 Days at
Various MgCl₂ Concentrations

| | 0.08 mM | 0.5 mM | 1 mM | 2 mM | 5 mM | 10 mM |
|---|---|---|---|---|---|---|
| | MgCl₂ | MgCl₂ | MgCl₂ | MgCl₂ | MgCl₂ | MgCl₂ |
| Relative % | 39 +/− 4 | 45 +/− 4 | 43 +/− 2 | 24 +/− 0.5 | 22 +/− 2 | 29 +/− 4 |
| Ad.SEAP | | | | | | |
| Activity | | | | | | |

The experimental results set forth in Table 3 demonstrate the importance of a divalent metal salt concentration of about 0.05-2 mM in a non-enveloped viral vector particle composition so as to maximize non-enveloped viral vector particle transgene expression level retention. Compositions comprising MgCl₂ in a concentration of more than 2 mM MgCl₂ exhibited significantly lower levels of relative transgene expression activity than compositions comprising between 0.08 mM and 1 mM MgCl₂ The best results as regards non-enveloped viral vector particle transgene expression level retention were observed for compositions comprising 0.5 mM and 1 mM MgCl₂.

The results of these experiments demonstrate that a composition having the desired divalent metal salt concentration provides for improved retention of non-enveloped viral vector particle transgene activity.

### Example 4

This example illustrates the stabilizing effects of arginine on non-enveloped viral vector particles when used in a composition of the invention.

Two solutions comprising sterile water, 10 mM Tris-HCl (pH of 7.8 at room temperature), 75 mM NaCl, 5% (wt./vol.) trehalose, and 0.08 mM MgCl₂ in equal volume were prepared. One of the solutions was modified by the addition of arginine to a concentration of 20 mM. An equal amount of Ad.SEAP particles, as described in Example 1, was added to each of the solutions. Aliquots of each solution were stored at −20° C., 4° C., or 37° C. in plastic containers for 1, 8, 28, and 70 days, and then the activity of the viral vector was determined. Activity was determined by measuring the amount of SEAP produced upon infection of A549 cells with a sample of each of the compositions. Experiments were repeated until a range of activity measurements was obtained. The results of these experiments are set forth in Table 4.

TABLE 4
Percent Relative Transgene Expression Activity of Composition
With or Without Arginine) Over Time

| | 4° C. Storage | | 25° C. Storage | | 37° C. Storage | |
|---|---|---|---|---|---|---|
| | no | arginine | no | arginine | no | arginine |
| | arginine | present | arginine | present | arginine | present |
| 1 day | nd | nd | Nd | nd | 73 | 91 |
| 8 days | nd | nd | 71 | 77 | 31 | 29 |
| 28 days | 81 | 97 | Nd | nd | nd | nd |
| 70 days | 51 | 75 | 15 | 14 | nd | nd |

nd = not determined

The experimental results set forth in Table 4 demonstrate that the presence of arginine in the composition of the invention has a significant stabilizing effect on the activity of non-enveloped viral vector particles maintained in the composition. The most significant difference was observed at 4° C. After 28 days, there was 16% more viral vector activity observed in the composition with 20 mM arginine than in the composition without arginine. After 70 days, there was 14% more viral vector activity in the composition with 20 mM arginine than in the composition without arginine. Similar results were observed after 7 days at 25° C., when there was 6% more viral vector activity in the composition with 20 mM arginine, and after 1 day at 37° C. when there was 18% more viral vector activity in the composition with 20 mM arginine, relative to the composition with no arginine.

These experimental results demonstrate that non-enveloped viral gene transfer vectors can be maintained in the composition of the invention for a sustained period of time while retaining a high level of transgene expression activity.

### Example 5

This example compares the non-enveloped viral vector particle activity-maintaining potential of alanine, histidine, and arginine, with either 20 mM NaCl or 75 mM NaCl, in a liquid composition over a period of four days.

Alanine, arginine, and histidine were added at differing concentrations to a composition comprising sterile water, 0.08 mM MgCl₂, 3% (wt./vol.) sucrose, 10 mM Tris-HCl (pH 7.8 at room temperature), and either 20 mM or 75 mM NaCl.

An equal amount of Ad.SEAP particles, as described in Example 1, was added to each of the solutions. Aliquots of each solution were stored at 25° C. or 37° C. in plastic containers for 4 days, and then the activity of the viral vector was determined. Activity was determined by measuring the amount of SEAP produced upon infection of A549 cells with a sample of each of the compositions. Experiments were repeated until a range of activity measurements was obtained. The results of these experiments are set forth in Table 5.

TABLE 5
Percent Activity of Compositions With Differing Amino Acid
Concentrations After 4 Day Incubation

| | 25° C. Storage | | 37° C. Storage | |
|---|---|---|---|---|
| | 20 mM NaCl | 75 mM NaCl | 20 mM NaCl | 75 mM NaCl |
| 10 mM | 0 | 2 | 0 | 7 |
| alanine | | | | |
| 20 mM | 0 | 3 | 0 | 7 |
| alanine | | | | |
| 40 mM | 0 | 2 | 0 | 10 |
| alanine | | | | |
| 10 mM | 2 | 64 | 3 | 19 |
| arginine | | | | |
| 20 mM | 19 | 79 | 16 | 57 |
| arginine | | | | |
| 40 mM | 84 | 87 | 60 | 61 |
| arginine | | | | |
| 5 mM | 1 | 26 | 1 | 9 |
| histidine | | | | |
| 15 mM | 0 | 7 | 0 | 15 |
| histidine | | | | |

As illustrated by the experimental results set forth in Table 5, the presence of arginine in the composition resulted in a significantly greater retention of transgene expression activity as compared to presence of alanine or histidine in the composition. For example, at 25° C. and 75 mM NaCl, the presence of 40 mM alanine in the composition resulted in the retention of only 2% transgene expression activity, and the presence of 5 mM histidine in the composition resulted in a retention of 26% transgene expression activity, while the presence of 40 mM of arginine in the composition resulted in the retention of 87% transgene expression activity. The ability of arginine to maintain high levels of transgene expression activity was more pronounced at higher arginine concentrations in the composition.

The experimental results set forth in Table 5 also illustrate the importance of the ionic strength of the composition on non-enveloped viral vector particle activity retention. In particular, a composition with an ionic strength of about 50-100 mM can provide an improved retention of non-enveloped viral vector particle activity over a sustained period of time. Moreover, the experimental results demonstrate the ability of an effective amount of arginine to complement the viral vector particle stability-promoting effects of the ionic strength of the composition. The composition comprising 20 mM NaCl and 40 mM arginine exhibited about four times greater transgene expression activity as compared to the composition comprising only 20 mM NaCl and 20 mM arginine at both 25° C. and 37° C.

The results of these experiments demonstrate that a composition comprising arginine provides for improved retention of non-enveloped viral vector particle transgene activity.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition for maintaining a non-enveloped viral vector comprising:
(a) about 1-25% (wt./vol.) trehalose,
(b) about 0.05-2 mM of a divalent metal salt, a cationic polymer, or a combination thereof,
(c) a multiplicity of non-enveloped viral vector particles, and
(d) a liquid carrier.

2. The composition of claim 1, wherein the composition comprises about 0.05-2 mM of a divalent metal salt.

3. The composition of claim 2, wherein the composition comprises about 0.05-2 mM MgCl₂.

4. The composition of claim 2, wherein the composition further comprises a nonionic surfactant in a concentration of about 0.001-0.015% (wt./vol.).

5. The composition of claim 3, wherein the nonionic surfactant is polysorbate 80.

6. The composition of claim 2, wherein the concentration of the multiplicity of non-enveloped viral vector particles is about 1×10⁵ to about 1×10¹³ FFU/ml.

7. The composition of claim 2, wherein the osmolality of the composition, in liquid form, is about 150-800 mOsM.

8. The composition of claim 2, wherein the ionic strength of the composition, in liquid form, is about 10-200 mM.

9. The composition of claim 2, wherein the composition further comprises a buffer, such that the pH of the composition is about 6 to about 9 when the temperature of the composition is about 25° C.

10. The composition of claim 2, wherein the composition further comprises about 10-65 mM arginine.

11. The composition of claim 1, wherein the non-enveloped viral vector is an adenoviral vector.

12. The composition of claim 10, wherein the adenoviral vector is replication-deficient.

13. The composition of claim 2, wherein the non-enveloped viral vector is an adenoviral vector.

14. The composition of claim 13, wherein the adenoviral vector is replication-deficient.

15. A method of preserving a non-enveloped viral vector comprising maintaining a multiplicity of non-enveloped viral vector particles in the liquid composition of claim 1 for a period of about 48 hours, wherein at least about 50% of the non-enveloped viral vector particles in the composition are active at the end of the period.

16. The method of claim 15, wherein the composition is maintained at a temperature of about 25° C. for the period of about 48 hours.

17. A method of preserving a non-enveloped viral vector comprising maintaining a multiplicity of non-enveloped viral vector particles in the liquid composition of claim 2 for a period of about 48 hours, wherein at least about 50% of the non-enveloped viral vector particles in the composition are active at the end of the period.

18. The method of claim 17, wherein the composition is maintained at a temperature of about 25° C. for the period of about 48 hours.

19. A method of administering a non-enveloped viral vector particle to a host cell comprising contacting a host cell with the liquid composition of claim 1 to infect the host cell with at least one non-enveloped viral vector particle.

20. A method of administering a non-enveloped viral vector particle to a host cell comprising contacting a host cell with the liquid composition of claim 2 to infect the host cell with at least one non-enveloped viral vector particle.

21. The method of claim 20, wherein the non-enveloped viral vector particles are recombinant viral vector particles comprising a transgene which is expressed in the host cell.

22. The method of claim 21, wherein the host cell is in a mammal.

23. The method of claim 22, wherein the mammal is a human.

24. The method of claim 23, wherein the host cell is in a heart.

25. The method of claim 23, wherein the non-enveloped viral vector is an adenoviral vector.

26. The method of claim 25, wherein the adenoviral vector is replication-deficient.
